(19) United States
(12) Patent Application Publication
(10) Pub. No.: US 2020/0011857 A1
(43) Pub. Date: Jan. 9, 2020

(54) SYSTEM AND METHOD FOR THERMAL CONTROL OF INCUBATION SYSTEM IN DIAGNOSTIC ANALYZER

(71) Applicant: Siemens Healthcare Diagnostics Inc., Tarrytown, NY (US)

(72) Inventors: Ayman Ismail, Tarrytown, NY (US); Eric Gerolstein, Melbourne, FL (US)

(73) Assignee: Siemens Healthcare Diagnostics Inc., Tarrytown, NY (US)

(21) Appl. No.: 16/492,926

(22) PCT Filed: Feb. 20, 2018
(86) PCT No.: PCT/US2018/018742

§ 371 Date: Sep. 10, 2019

Related U.S. Application Data
(60) Provisional application No. 62/472,472, filed on Mar. 16, 2017.

(51) Int. Cl. G01N 33/53; H04L 1/16; G01N 35/02

(52) U.S. Cl. CPC G01N 33/53; G01N 2035/00376; G01N 35/021; H04L 1/1671

(57) ABSTRACT

An incubation system for use in an in vitro diagnostic analyzer utilizes a fixed base and a rotatable ring that holds reaction cuvettes. Thermal regulation is provided by directly mounting a heating element to the rotatable ring and controlling the temperature using a temperature controller and thermal sensor for detecting the temperature of the ring, either of which may be mounted to the ring.

### CROSS-REFERENCE TO RELATED APPLICATIONS

This application claims priority to U.S. provisional application Ser. No. 62/472,472 filed on Mar. 16, 2017, the contents of which is incorporated herein by reference in its entirety.

### BACKGROUND

In vitro diagnostics (IVD) allows labs to assist in the diagnosis of disease based on assays performed on patient fluid samples. IVD includes various types of analytical tests and assays related to patient diagnosis and therapy that can be performed by analysis of a liquid sample taken from a patient's bodily fluids, or abscesses. These assays are typically conducted with automated immunoassay analyzers or clinical chemistry analyzers (analyzers) onto which fluid containers, such as tubes or vials, containing patient samples have been loaded. The analyzer extracts a liquid sample from the vial and combines the sample with various reagents in special reaction cuvettes or tubes (referred to, generally, as reaction vessels).

A modular approach is often used for analyzers. Some larger systems include a lab automation system that can shuttle patient samples between one sample processing module and another module. These modules include one or more stations, including sample handling stations and testing stations. Testing stations are units that specialize in certain types of assays and provide predefined testing services to samples in the analyzer. Exemplary testing stations include immunoassay (IA) and clinical chemistry (CC) stations. In some laboratories, typically including smaller labs, these testing stations can be provided as independent/standalone analyzers or testing modules, allowing an operator to manually load and unload individual samples or trays of samples for CC or IA testing at each station in the lab.

A typical IA analyzer module is a clinical analyzer (integrated into a larger analyzer or standing alone) that automates heterogeneous immunoassays using magnetic separation and chemiluminescence readout. Immunoassays take advantage of the existence of either specific antibodies for the analytes being tested or specific antigens for the antibodies being tested. Such antibodies will bond with the analyte in the patient's sample to form an “immune complex.” In order to use antibodies in immunoassays, they are modified in specific ways to suit the needs of the assay. In heterogeneous immunoassays, one antibody (capture antibody) is bound to a solid phase, a fine suspension of magnetic particles for the IA module, to allow separation using a magnetic field followed by a wash process. This is exemplified in sandwich assays and competitive assays. An exemplary IA module menu can include additional variations on these formats.

In the typical sandwich assay format, two antibodies are used, each one selected to bind to a different binding site on the analyte's molecule, which is usually a protein. One antibody is conjugated to the magnetic particles. The other antibody is conjugated to an acridinium ester molecule (AE). During the assay, sample and the two modified antibody reagents are added to a cuvette. If the analyte is present in the patient's sample, the two modified antibodies will bind and “sandwich” the analyte molecule. Then, a magnetic field is applied which will attract the magnetic particles to the wall of the cuvette, and excess reagents are washed off. The only AE-tagged antibody left in the cuvette is one that formed an immune complex through the sandwich formation with the magnetic particles. Acid solution is then added to free up the AE into solution, which also includes hydrogen peroxide needed for the chemiluminescence reaction. A base is then added to cause it to decompose, emitting light (see reaction formulas below—a variety of AEs are used in various assays but the fundamental chemistry is substantially identical). Light is emitted as a flash lasting a few seconds and is collected and measured in a luminometer. The integrated light output is expressed as relative light units (RLU's). This is compared to a standard curve which is generated by fitting a dose-response curve to RLU values generated by known standards of the same analyte over its clinical range. Sandwich assays produce a direct dose-response curve where higher analyte doses correspond to increased RLUs.

The competitive assay format applies to molecules for which only one antibody is used. This antibody is conjugated to the magnetic particles. A second assay reagent contains the analyte molecule conjugated to the AE. During the assay, the quantities of the reagents are chosen such that the analyte from the patient's sample and the AE-tagged analyte compete for a limited amount of the antibody. The more patient analyte there is, the less AE-tagged analyte will bind to the antibody. After magnetic separation and wash, the only source of AE in the cuvette is from AE-tagged analyte that has been bound to the magnetic particles through the antibody. Acid and base are added as before, and the dose analysis is as described for the sandwich assay. Competitive assays produce an inverse dose-response curve, where a higher signal corresponds to a lower amount of analyte in the patient sample.

The IA analyzer module magnetic particle reagent is also referred to as the “solid phase” and the AE-tagged reagent is referred to as the “lite reagent.” The IA analyzer module provides the hardware and software to enable running multiple assays of various formats concurrently in random-access and with high throughput.

At the heart of a typical IA analyzer/module is an incubation ring. To perform the above-described assays, the reactions need to take place at a well-controlled temperature range, typically coinciding with nominal temperature of the human body. An incubation ring provides a regulated thermal body to ensure that cuvettes maintain this temperature range while the cuvettes move in the IA module. By providing a ring, random access to cuvettes can be provided. This allows assays of varying length to be performed in parallel, allowing some cuvettes to receive analytes/reagents, some receive sample aliquots, some to be analyzed, some to be washed, etc. simultaneously. The ring can then be moved at regular intervals under processor control to ensure that reactions take place at a controlled incubation temperature for a prescribed time interval before analysis of the reaction.

The incubator ring rotates relative to a fixed base, typically driven by a motor affixed to the base that drives a gear ring or belt on the moving ring. To regulate temperature, the base is commonly heated with a conventional heating element driven by a controller that receives thermal feedback from a temperature sensor in thermal contact with the base. The thermally controlled base heats the air gap between the base and the incubator ring, which heats the cuvette. An enclosure is provided to help insulate the entire volume of air inside. By residing in the thermally regulated air, the ring maintains the set temperature when in a steady state condition. However, the transient response of this set up is not ideally damped and has substantial thermal lag as the ring is heated indirectly via the surrounding air and air gap between the heated base and the ring. Additionally, by providing a heated base, to maintain thermal uniformity, the base is typically made from metal, such as machined aluminum, which increases the cost of the base structure.

### BRIEF DESCRIPTION OF THE FIGURES

FIG. 1 is a perspective view of an exemplary incubation ring system for use with some embodiments;

FIG. 2 is an overhead view of an exemplary incubation ring system for use with some embodiments;

FIG. 3 is a cross sectional view of an exemplary incubation ring system for use with some embodiments;

FIG. 4 is a perspective cross-sectional view of an exemplary incubation ring system for use with some embodiments; and

FIG. 5 is a perspective cross-sectional view of an exemplary incubation ring system for use with some embodiments.

### DETAILED DESCRIPTION OF THE EMBODIMENTS

Embodiments of an incubation ring utilize a heating element directly in thermal contact with the rotatable body of the ring to overcome problems with thermal lag in the control loop for regulating that temperature of the incubation ring. Reaction cuvettes are placed in slots in the incubation ring and heated by contact with the ring (and by conductive, radiant, and convective processes by sitting in the slots in contact with the ring or in close proximity where there is any small air gap between the cuvette and slot of the ring). A slot is any orifice designed to accept a cuvette and hold it securely in the incubation ring. By placing the heating element directly in contact with the rotatable ring, the heating element can more directly heat the cuvettes to a controlled temperature (e.g., approximately body temperature) than traditional incubation rings that rely on indirectly heating the incubation ring (e.g., by heating the fixed base that is separated by an air gap from the ring, effectively creating a low-temperature oven, transferring heat via the air gap).

Prior art systems that heat the static base have an air gap between the heated stationary base and the rotating incubation ring that holds cuvettes. That is, the moving ring is parasitically coupled to the basin by a small air gap that creates an under-damped system. In the case of overshooting the set point temperature at the base, the system is limited in restoring thermal equilibrium, and therefore the optimal performance is hindered. One way to address this issue is to measure the temperature of the base, while heating the base. However, this simply creates an oven effect, whereby the stationary base heats the internal air cavity of the incubation system. Meanwhile, empty cuvettes within the moving incubation ring start at a certain temperature, such as an ambient temperature outside of the incubation system. Sample aliquots and refrigerated reagents are dispensed into these cuvettes. This results in thermal lag between the incubation ring and the stationary base. Thus, heating and measuring the stationary base and parasitically heating the incubation ring results in varying inaccuracies in the temperature of the ring.

Applying a different measurement technique to the moving ring, while heating of the stationary base, does not solve this issue. For example, where a non-contact IR sensor is used to measure the temperature of the moving ring, the air gap between heated base of the moving ring still results in a delay, which can cause the system to be under-damped or over-damped. If under-damped, the non-optimum response of the system results in a ring, whereby the temperature overshoots the set point, causing the ring to be too hot for a certain time, which is undesirable in an incubation system. To address this, one could add a cooling function to the heat source or use a more complex predictive control scheme that is not suitable for random-access throughput of an incubation system.

Embodiments overcome these control loop in thermal lag problems by moving the heating element from the traditional placement on the stationary base to the rotatable body of the incubation ring. Then, by measuring the temperature of the incubation ring, control loop delay can be removed. Because the incubation ring rotates, moving heating element to the rotating member presents certain electrical problems. Namely, electrical current used to heat the heating element must be applied to the rotating ring. Similarly, in embodiments where the temperature measurement of the ring occurs via a thermally contacted sensor on that ring, and where that sensor produces a DC signal, that signal should be conducted from the rotating ring to a controller.

Embodiments address the issue of transmitting current to the heating element and sensor readings from the rotating ring to the controller by using one or more slip rings. A slip ring is an electromechanical device that allows the transmission of power and electrical signals from a stationary structure to a rotating structure. A slip ring can be used in electromechanical systems that requires rotation while transmitting power or signals. Also called rotary electrical interfaces, rotating electrical connectors, collectors, swivels, or electrical rotary joints, these rings are commonly found in slip ring motors, electrical generators for alternating current (AC) systems and alternators, packaging machinery, cable reels, and wind turbines. They can be used on a rotating object to transfer power, control signals, or analog or digital signals. Typically, a slip ring consists of a stationary graphite or metal contact (brush), which rubs on the outside diameter of a rotating metal ring. As the metal ring turns, the electric current or signal is conducted through the stationary brush to the metal ring making the connection. Additional ring/brush assemblies are stacked along the rotating axis if more than one electrical circuit is needed. Either the brushes or the rings are stationary and the other component rotates.

In some embodiments, multiple slip rings are used. For example, the body of the rotating incubator ring can be grounded via ball bearings (or via a slip ring) between the static base and the incubator ring. A first slip ring it can transmit electrical current at the direction of the thermal controller to heat a heating element mounted directly to the incubator ring, such that the heating element is in direct thermal contact with the incubator ring and powered via the slip ring. A second slip ring can be used to transmit the sensor signal from a thermal sensor mounted in direct thermal contact with the incubator ring. In some embodiments, multiple thermal sensors are mounted directly to the incubator ring, each using a slip ring to transmit the sensor signal. In some embodiments, the slip ring is used to transmit the ground signal. In some embodiments, inductive coupling is used to transmit control or power signals.

In some embodiments, the thermal controller is mounted directly to the rotating incubator ring, minimizing the number of slip rings needed to transmit signals. For example, a slip ring that can be used for a ground signal, power, and a configuration serial interface, such as an I²C interface. Meanwhile, in these embodiments, the heating element and one or more temperature sensors are directly mounted to the rotating incubator ring, along with any control circuitry and the thermal controller. While this increases the complexity of the incubator ring, the complexity is removed from the static base and the performance of the thermal feedback loop is improved over the prior art systems that heat and monitor the static base, rather than the rotating incubator ring.

In some embodiments, the heating element is a flexible heater to allow the heating element to be substantially uniformly applied to the circumference of the rotating incubation ring. A flexible heater is a device to conform to the surface, which requires heating, and generally takes electrical power to convert to heat. For example, a flexible heating element can be wrapped around the outside of the incubation ring such that more than 95% of the circumference has a portion of the heating element applied to it via an adhesive. That is, approximately the entire circumference receives thermal energy uniformly when the heating element is energized. The outer circumference or inner circumference of the incubation ring is generally cylindrical. While the axial dimension of that cylinder need not necessarily have uniform heating, it is ideal to uniformly apply heat in the circumferential dimension of that cylinder (e.g. a heater band that is less wide than the cylinder, but which covers 95%). By using a flexible heater (or a heater molded to match the circumference of the incubation ring), the heater can be mounted to be in uniform thermal contact with the body of the ring without air gaps. In some embodiments, a good conductor of heat, such as aluminum, can be used as the material for the incubation ring to mitigate any thermal non-uniformities, allowing uniform heating to cuvettes housed in the incubation ring.

Exemplary flexible heating elements include a conductor, such as a metal coil or foil that can optionally be wrapped in a temperature-stable polymer. In some embodiments, polyimide foil embedded in DuPont Kapton polymer film provides a flexible heating element that can easily by applied to the circumference of the incubation ring with an appropriate adhesive. Some embodiments utilize a heater of metallic film embedded in fiber-reinforced silicone.

In some embodiments, an onboard temperature controller is provided in a small controller board package that is approximately 4 cm×4 cm×5 mm. This lightweight control board can be placed easily within the incubation ring body or mounted thereto. Temperature readings used by this controller board can be provided by thermistors. Thermistors are inexpensive, fairly accurate, pre-calibrated temperature sensors that can be used to measure temperature of a contacting surface. By placing the temperature controller board on or in the incubation ring body, multiple thermistors can be used without increasing the number of electric slip rings used to carry sensor signals. By directly wiring thermistors to a control board on the rotating ring, noise that may be caused by rotation of the electric slip ring can be avoided in the sensor signals.

In some embodiments, the temperature controller is mounted to the static base and control signals for energizing heater are transmitted from the controller to the flexible heater via the electric slip rings. In these embodiments, the temperature signal can be created by thermistors or other thermal sensors mounted directly to the rotating incubator ring via slip rings. In some embodiments, the thermal sensing is done via an IR, noncontact method. An IR sensor can view the IR range emitted by the rotating incubator ring and translate this to a temperature. The IR sensor can be placed on the static base, allowing the sensor signal to be hardwired to the temperature controller without the need of an intervening slip ring.

Advantages of placing the heater directly in thermal contact with the rotating incubator ring, rather than by heating the static base, can include the following. A simplified heat transfer path is created that avoids the air gap between the base and rotating ring. The need to heat the base is eliminated by decoupling that base from the thermal control loop. This can allow different choice of materials (e.g., molded plastic rather than aluminum) and designs the static base. Furthermore, because the base does not need to be heated, a lower watt density heater that consumes less power may be used. Generally, the incubation system will reach prescribed temperatures more quickly due to the direct contact of the heater in proximity to the temperature sensor. This also ensures that cuvettes heat up more quickly when a colder sample is placed into a cuvette it incubation ring. By placing the thermal controller directly in the rotating ring, a more compact and less complex control module can be used, without requiring additional wiring. The incubation ring can be assembled a self-contained object with the only electrical interface being the slip rings, reducing the electrical complexity of the static base.

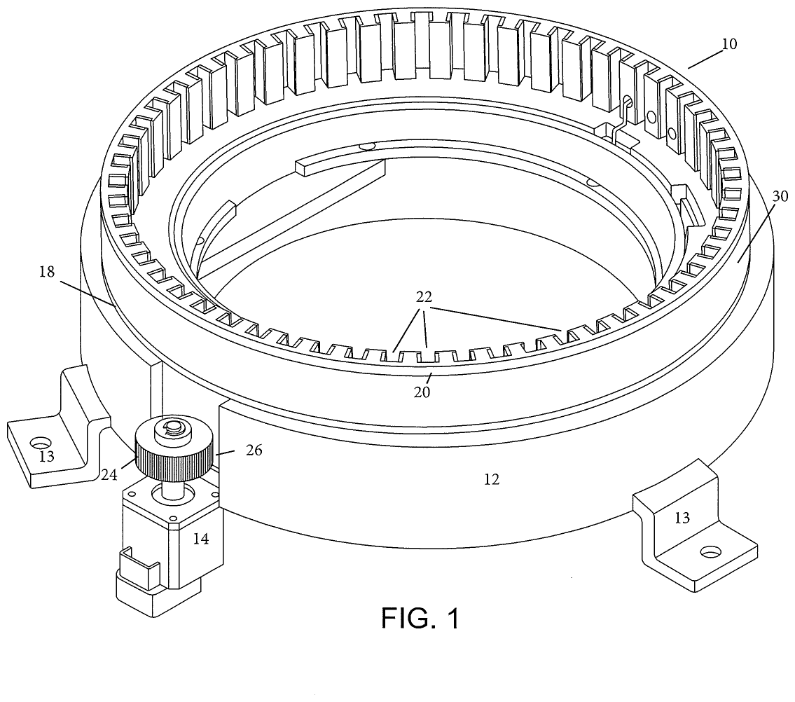

FIG. 1 is a perspective drawing of an exemplary incubation ring 10 and static base 12. Static base includes flanges 13 for mounting to an immunoanalyzer system. Motor 14 (e.g., a stepper motor) drives the incubation ring 10, which sits on top of the static base 12 in a ring-shaped recess 18 of that base. The incubation ring 10 includes a body 20 of machined or molded aluminum having a plurality of open slots 22 around the inner circumference of the incubation ring. The slots 22 are designed to interface rectangular-shaped cuvettes. These cuvettes are made of a sterile plastic material and are disposable. In some embodiments, the slots used are only open at the top and can be of any suitable shape. Motor 14 moves a gear 24 that interfaces a circumferential ring gear on the incubation ring to move the ring for random access to cuvettes. Pipettes can then access cuvettes that are moved to a certain location in the ring via the motor. The system in FIG. 1 (and the other figures) is typically placed inside of an enclosure to assist in thermal stability. A heating element 30 is wrapped around the outside of circumference of the incubation ring body. The thermal controller is affixed to the underside of the incubation ring, along with a plurality of thermistors placed at equal spacing around the incubation ring in thermal contact with the body of each ring. Obscured in this view are multiple electrical slip rings to provide power, ground, and configuration signaling to the thermal controller in the incubation ring.

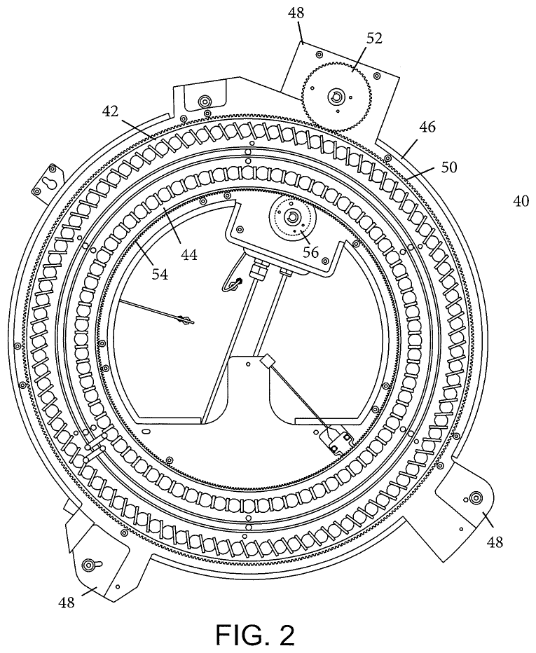

FIG. 2 is a top view of an alternate embodiment of an incubation system 40. In this embodiment, a pair of incubation rings 42 and 44 are provided. A larger radius 42 and smaller radius incubation ring 44 are provided concentrically. This roughly doubles the capacity of the incubation system with approximately the same footprint. Like the system shown in FIG. 1, a static base 46 has a means for allowing it to be mounted in an immunoanalyzer (e.g., via flanges 48 or an adapter plate). A ring gear 50 is placed on the outside of the body of the outside incubation ring 42 and interfaces the drive gear 52 of a stepper motor mounted to the static base 46. A ring gear 54 is also mounted to the inside circumference of the body of the inner incubation ring and driven by a separate stepper motor gear 56 mounted to the static base. This allows random access to either of the incubation rings, allowing rings to rotate for access to cuvettes by various stations in pipettes that interface the rings. Each ring includes a plurality round slots that are configured to accept appropriately-shaped cuvettes. Heating elements and thermal controllers are placed on each of these incubation rings is described with respect to FIG. 1.

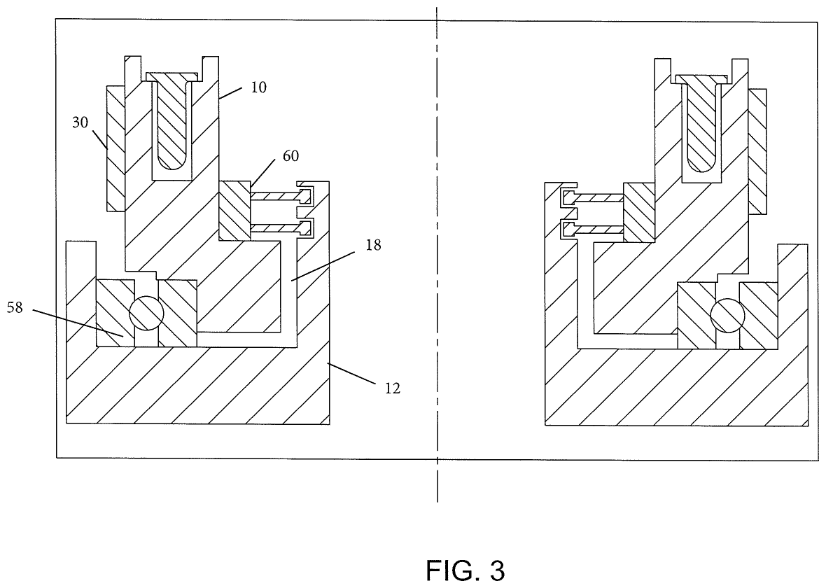

FIG. 3 is a cutaway view of an exemplary incubation system illustrating the interface between the static base 12 and the incubation ring 10 that rotates about the static base. The static base includes a ring-shaped recess 18 in which the incubation ring rides. A ring-shaped ball bearing 58 provides a low friction means for the incubation ring 10 to rotate within this recess 18. Also within this recess is a pair of electrical slip rings 60 that provide at least power and ground signals to the incubation ring. On the outside circumference of the ring, the flexible heating element 30 is wrapped substantially around the entire circumference to provide uniform heating.

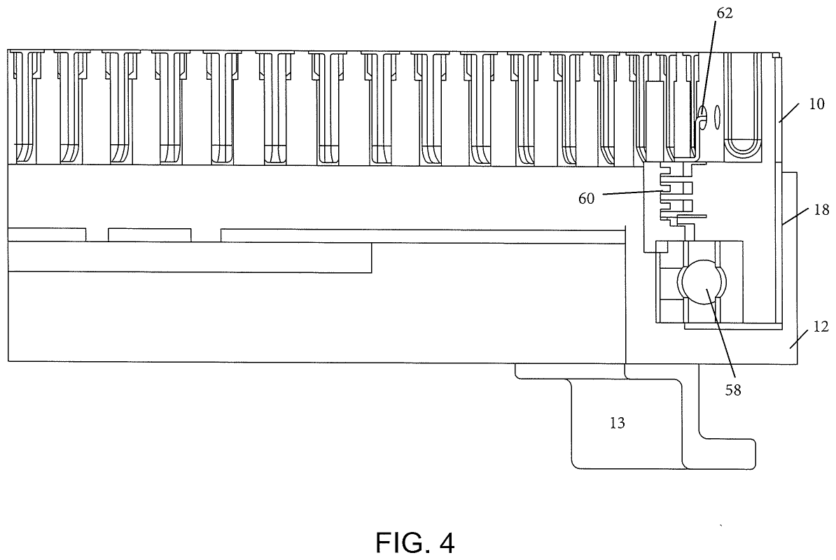

FIG. 4 is a cutaway perspective view of the embodiment shown in FIG. 1. Like the example shown in FIG. 3, the static base 12 includes a ring-shaped in which the incubation ring 10 rides. The ball bearing 58 provides lubrication and allows low friction rotation of the incubation ring. In this example, three electrical slip rings 60 are provided within that recess to allow configuration, power, and ground signals to be provided from circuitry within the remainder of the immunoanalyzer or the base, including a processor that manages the entire system and configures the thermal controller. A plurality of thermistors 62 are mounted to the ring 10. This control can include setting a target temperature, setting PID values, setting threshold values, etc. Exemplary thermal controllers can include PID controllers or threshold-based/bang-bang controllers to maintain temperature within a given range. In some embodiments, PID control is preferred, either circuit-based or software-based.

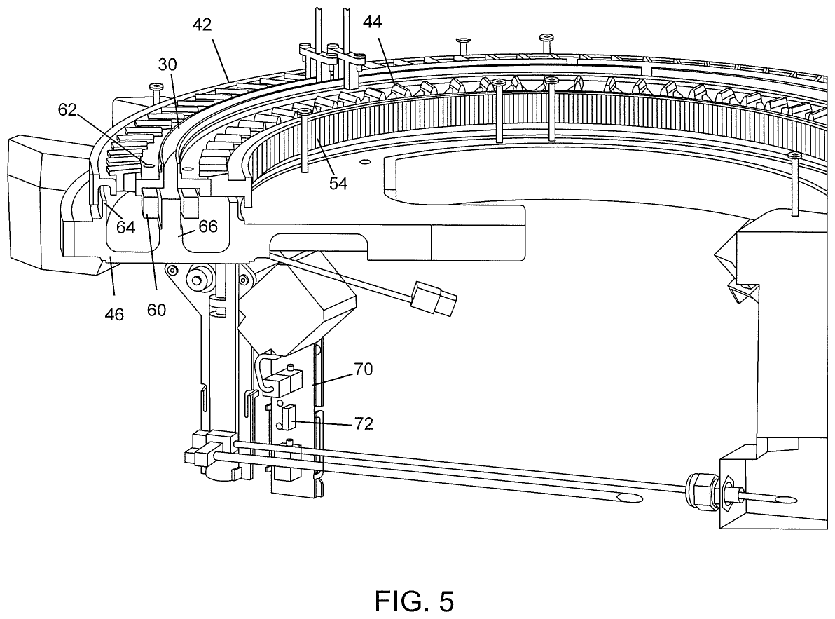

FIG. 5 is a three-dimensional cutaway view of the embodiment shown in FIG. 2. In this embodiment, two concentric incubation rings 42 and 44 ride atop a plurality of lips 64 provided by the static base 46. These lips act as rails to allow the controlled rotation of the two rings. Recesses within the static base 46 allow the bottoms of the cuvettes to pass by without obstruction. The plurality of electrical slip rings 60 on a rail 66 spaced between the two rings allows for signaling to be passed from circuitry within the base to the thermal controller within each ring. A heating element 30 is placed on the inner circumference and outer circumference of the body of each respective ring. A plurality of thermistors 62 are mounted to each ring. In this illustrated embodiment, a control board 70 is mounted to the underside of the base that includes communications interfaces for interfacing to the rest of the immunoanalyzer, motor controllers, and a thermal controller 72 for regulating temperature of the incubation rings. In some embodiments, thermal controller 72 is mounted directly to the body of each incubation ring 42 and 44 and communicates with board 70 via the electrical slip rings.

An exemplary method for providing thermal control to an incubator system within an immunoanalyzer includes providing at least one incubation ring that has a heating element placed about the circumference of that ring. The controller is provided within that ring for regulating the temperature of that ring by selective activation of the heating element. Power and signaling is provided to the controller from a static base via electrical slip rings. In some embodiments, signaling to configure the controller is provided wirelessly. The temperature controller is configured to sense at least a predetermined temperature set point for that incubation ring. The temperature controller then monitors the one or more of thermistors or other temperature sensors that are also thermally mounted to that ring. The temperature controller provides these thermal readings, such as an average of these readings, to a control circuit to provide a feedback signal. For example, an average of thermistor values is provided to a potential, an integral, and a differential circuit (or virtualized in software) and a weighted sum of the outputs of these circuits is provided as a control signal to an output that drives a heating element. This provides PID control. The control signal is then provided to the heating element to provide a current that heats up the heating element on demand. This control loop continues to regulate the temperature by constantly adjusting the output to the heating element based on the temperature sensor readings.

What is claimed is:

1. A thermally regulated incubator for use in an immunoassay, comprising:
a fixed base;
an incubation ring comprising a body configured to hold a plurality of cuvettes, rotatably coupled to the fixed base;
a heating element thermally affixed to the incubation ring, such that the heating element rotates with the incubation ring;
at least one thermal sensor configured to directly measure a temperature of the incubation ring; and
a thermal controller configured to control the heating element in response to real time measurement of the at least one thermal sensor to maintain the incubation ring within a predetermined temperature range.

2. The thermally regulated incubator of claim 1, wherein the heating element is a flexible heating element comprising a conductive material and a polymer material.

3. The thermally regulated incubator of claim 1, wherein the thermal controller is mounted to the incubation ring.

4. The thermally regulated incubator of claim 1, further comprising electrical slip rings configured to conduct power from the fixed base to the incubation ring.

5. The thermally regulated incubator of claim 4, wherein the thermal controller is mounted to the fixed base and at least one slip ring is configured to conduct an electrical signal from the thermal controller to the heating element.

6. The thermally regulated incubator of claim 1, wherein the at least one thermal sensor is mounted in thermal contact with a surface of the incubation ring.

7. A thermally regulated incubator for use in an immunoassay, comprising:
a fixed base;
an incubation ring, rotatably coupled to the fixed base, comprising:
a plurality of slots configured to hold a plurality of cuvettes,
one or more temperature sensors configured to sense a temperature of the incubation ring, and
a heating element thermally affixed to the incubation ring in thermal contact with substantially the entire circumference of the incubation ring; and
a thermal controller configured to control the heating element to maintain the incubation ring within a predetermined temperature range.

8. The thermally regulated incubator of claim 7, wherein the heating element is a flexible heating element comprising a conductive material and a polymer material.

9. The thermally regulated incubator of claim 7, wherein the thermal controller is mounted to the incubation ring.

10. The thermally regulated incubator of claim 7, further comprising electrical slip rings configured to conduct power from the fixed base to the incubation ring.

11. The thermally regulated incubator of claim 10, wherein the thermal controller is mounted to the fixed base and at least one slip ring is configured to conduct an electrical signal from the thermal controller to the heating element.

12. The thermally regulated incubator of claim 7, further comprising a second incubation ring having one or more temperature sensors and a second heating element in thermal contact with substantially the entire circumference of the second incubation ring.
